(19) United States
(12) Patent Application Publication
(10) Pub. No.: US 2022/0055752 A1
(43) Pub. Date: Feb. 24, 2022

(54) HEALTH MONITORING SEATING SYSTEM

(71) Applicants: Goodrich Aerospace Services Private Limited, Bengaluru (IN); B/E Aerospace, Inc., Winston Salem, NC (US)

(72) Inventors: Sanith Kurumpilavu Subramanian, Chikkabanavara (IN); Dharamveer Surya Prakash Bathla, Sonepat (IN); Raja Mandava, Bangalore (IN); Nagesh Sadashivaiah, Kolar (IN); Vishwanand Vishwannathan, Bangalore (IN)

(73) Assignees: Goodrich Aerospace Services Private Limited, Bengaluru (IN); B/E Aerospace, Inc., Winston Salem, NC (US)

(21) Appl. No.: 17/399,186

(22) Filed: Aug. 11, 2021

(30) Foreign Application Priority Data
Aug. 17, 2020 (IN) 202041035388

(51) Int. Cl. B64D 11/00; B64D 11/06; A61B 5/0205; A61B 5/024; G06K 9/00

(52) U.S. Cl. CPC B64D 11/00155; B64D 11/0646; G06K 9/00362; A61B 5/024; A61B 5/02055

(57) ABSTRACT

A system includes a passenger seat. A biometric sensor is configured to monitor at least one biometric of a passenger seated in the passenger seat. A controller is operatively connected to control the biometric sensor and to provide output indicative of a health condition of the passenger based on the biometric.

### CROSS-REFERENCE TO RELATED APPLICATIONS

This application claims priority to Indian Provisional Patent Application No 202041035388, filed Aug. 17, 2020 the disclosure of which is incorporated by reference herein in its entirety.

### BACKGROUND

### 1. Field

The present disclosure relates to aviation, aircraft interiors, and pandemic control, and more particularly to seating systems in aircraft for monitoring health of passengers.

### 2. Description of Related Art

The COVID-19 outbreak has increased the need to control the transit of infected individuals as well as the asymptomatic individuals carrying a viral charge. Due to the proximity of passengers to one another during air travel, and due to the duration of spending time together in that proximity, there is a particular need to make air travel as safe as possible from an epidemiological perspective.

The conventional techniques have been considered satisfactory for their intended purpose. However, there is an ever present need for improved systems and methods for monitoring the health of individuals during air travel. This disclosure provides a solution for this need.

### SUMMARY

A system includes a passenger seat. A biometric sensor is configured to monitor at least one biometric of a passenger seated in the passenger seat. A controller is operatively connected to control the biometric sensor and to provide output indicative of a health condition of the passenger based on the biometric.

The passenger seat can include an armrest. The biometric sensor can be mounted in the armrest. A second passenger seat can be in front of the first passenger seat. The biometric sensor can include an imaging device mounted to the second passenger seat, facing back toward the first passenger seat, configured to monitor the first passenger seat for occupancy and temperature of occupant. The first passenger seat can include an armrest. A pulse oximeter can be mounted in the armrest. The pulse oximeter can be operatively connected to the controller to provide input relating to an occupant's pulse rate, heart rate, blood pressure, oxygen levels and/or vascular state. The controller can be configured to use the input to provide output indicative of an occupant's health state. A display can be mounted in the headrest of the second passenger seat, facing toward the first passenger seat, and operatively connected to the controller to output information regarding the occupant's health state. The armrest can include a display operatively connected to the controller to output information regarding the occupant's health state. The first and second passenger seats can be in a plurality of passenger seats, each having a respective pulse oximeter and a respective imaging device connected to a controller. A central display can be operatively connected to the pulse oximeters and imaging devices to alert a crewmember of a health status of any occupant of the passenger seats.

A method includes checking a seat for occupancy. Upon detecting occupancy in the passenger seat, the method includes monitoring an occupant in the passenger seat for one or more biometrics indicative of a health condition.

Monitoring can include monitoring temperature of the occupant and outputting an alert if the temperature of the occupant is outside of a predetermined range. Monitoring can include monitoring images of the occupant and outputting an alert if the images are indicative of a respiratory tract infection. Monitoring can include monitoring pulse rate of the occupant and outputting an alert if the pulse rate is outside of a predetermined range. Monitoring can include monitoring blood oxygen level of the occupant and outputting an alert if the blood oxygen level is outside of a predetermined range. The method can include outputting an alert to take quarantine measures for the occupant if the one or more biometrics is/are indicative of a contagious condition.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is a schematic perspective view of an embodiment of a system constructed in accordance with the present disclosure, showing first and second passenger seats, with an occupant in the first passenger seat being monitored by a biometric sensor in the second passenger seat;

FIG. 2 is a schematic side elevation view of a biometric sensor for the system of FIG. 1, showing a pulse oximeter mounted in the armrest of one of the passenger seats;

FIG. 3 is a schematic view of a synoptic display for the system of FIG. 1; and

FIG. 4 is a flow diagram of the system of FIG. 1, showing a method of monitoring heath using the system of FIG. 1.

### DETAILED DESCRIPTION OF THE PREFERRED EMBODIMENTS

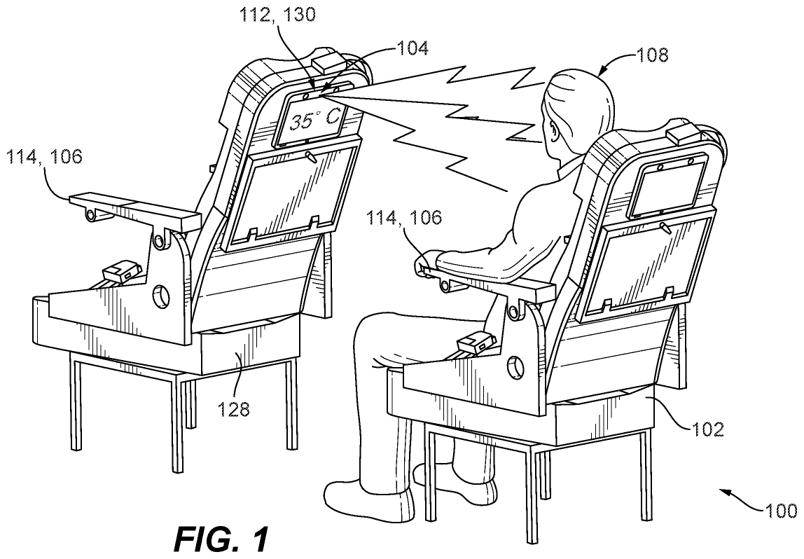

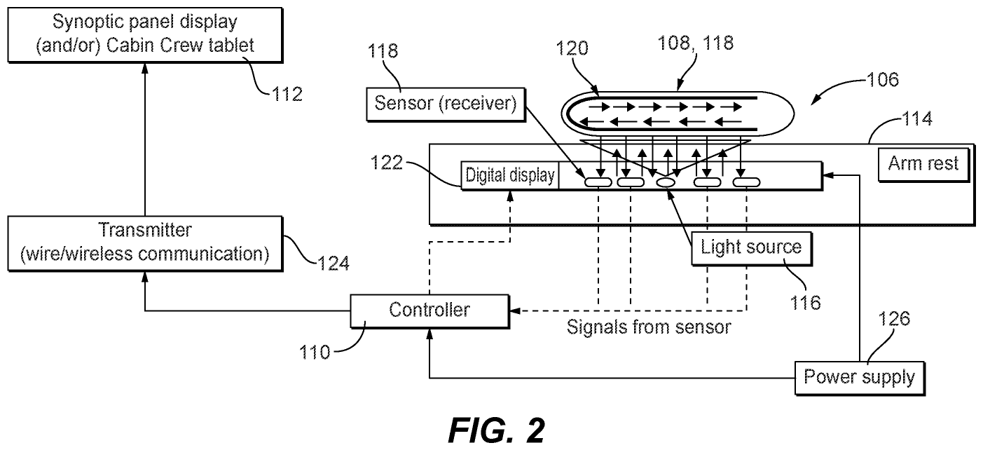

Reference will now be made to the drawings wherein like reference numerals identify similar structural features or aspects of the subject disclosure. For purposes of explanation and illustration, and not limitation, a partial view of an embodiment of a system in accordance with the disclosure is shown in FIG. 1 and is designated generally by reference character 100. Other embodiments of systems in accordance with the disclosure, or aspects thereof, are provided in FIGS. 2-4, as will be described. The systems and methods described herein can be used to monitor health of passengers in transit.

The system 100 includes a first passenger seat 102. Biometric sensors 104, 106 are configured to monitor at least one biometric of a passenger 108 seated in the passenger seat 102. A controller 110 (labeled in FIG. 2) is operatively connected to control the biometric sensors 104, 106 and to provide output 112 indicative of a health condition of the passenger 108 based on the biometrics.

The passenger seat 102 includes an armrest 114. With reference to FIG. 2, the biometric sensor 106 includes a pulse oximeter mounted in the armrest 114. The pulse oximeter includes a light source 116 and receivers 118 is operatively connected to the controller 110 to provide input to the controller 110 relating to an seat occupant's pulse rate, heart rate, blood pressure, oxygen levels and/or vascular state, e.g. by scanning the finger 118 of the passenger 108 for blood flow 120. The controller 110 is configured to use the input to provide output 112 indicative of an occupant's health state. The armrest 114 includes a display 122 operatively connected to the controller 110 to output information regarding the occupant's health state. This information can also be output on a synoptic display, e.g. on an IFE as described below, or on a tablet for the flight crew, as also described below. A wired and/or wireless transmitter 124 can connect between the controller 110 and the devices displaying the output 112. A power supply 126 is connected to power the biometric sensor 106, controller 110, and transmitter 124.

With reference again to FIG. 1, a second passenger seat 128 is in front of the first passenger seat 102. Another biometric sensor 104 includes an imaging device mounted to the second passenger seat 128, facing back toward the first passenger seat 102, configured to monitor the first passenger seat 102 for occupancy and for temperature of the occupant or passenger 108. The imaging device can be configured to use visible or infrared image data to detect whether or not there is a passenger or occupant 108 in the first passenger seat 102. The imaging device can also include thermal imaging capability for use in thermal imaging the occupant 108 to detect temperature regularities indicative of respiratory tract infection, for example. An IFE (in-flight entertainment) display 130 is mounted in the headrest of the second passenger seat 128, facing toward the first passenger seat 102 for viewing by the occupant 108 seated in the first passenger seat 102. The display 130 is operatively connected to the controller 110 (shown in FIG. 2) to output information regarding the health state of the passenger or occupant 108. Those skilled in the art will readily appreciate that the first and second passenger seats 102, 128 can be just two in a plurality of similar passenger seats, each having a respective pulse oximeter 106 and a respective imaging device 104 connected to a controller 110.

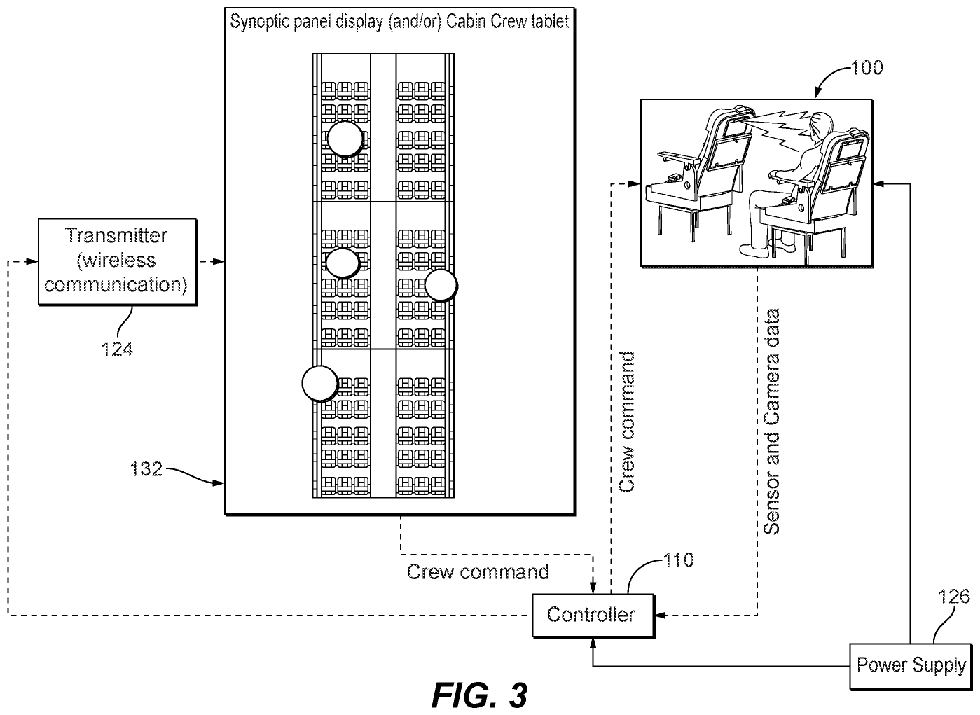

With reference now to FIG. 3, a central display, e.g., a tablet display 132 is operatively connected to the pulse oximeters 106 and imaging devices 104 (of FIG. 1) to alert a crewmember of a health status of any occupant of the passenger seats 102, 128. The view shown on the display 132 in FIG. 3 shows a floor plan of an aircraft cabin, highlighting the seats of passengers with potential health issues. The crew can issue commands from the tablet to the controller 110, e.g. to take quarantine measures such as altering air flow in the area of a cabin near passengers with potential contagions.

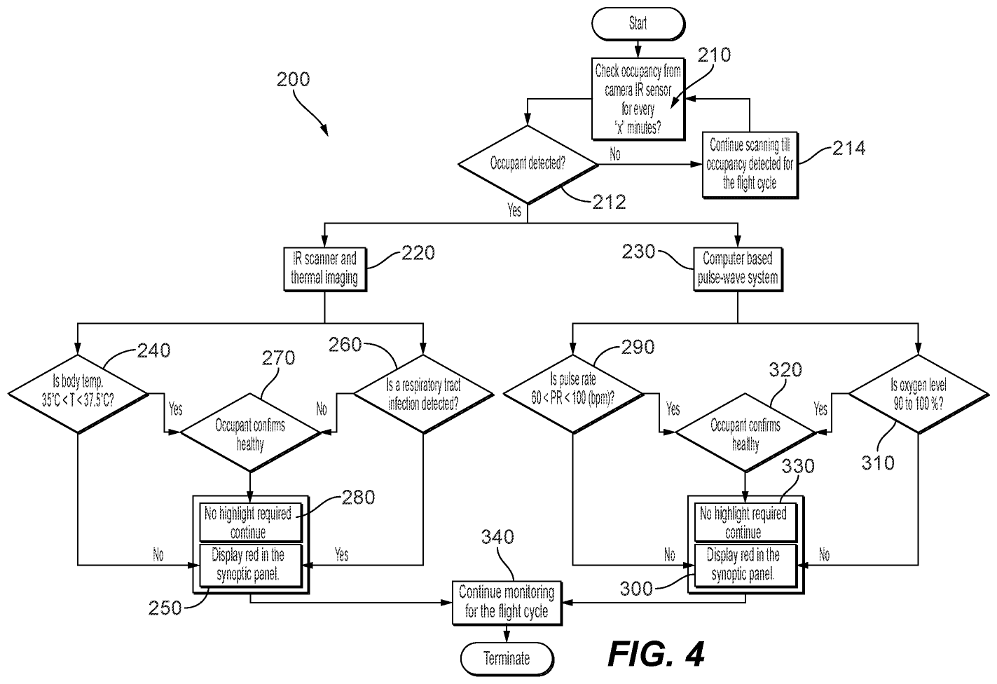

With reference now to FIG. 4, a method indicated by reference character 200 includes checking a seat for occupancy as indicated in box 210. This can be accomplished, e.g. using the IR capabilities of the sensor 104 described above. The check for seat occupancy can continue periodically in the event no passenger or occupant is detected, as indicated in FIG. 4 with boxes 212 and 214. Upon detecting occupancy in the passenger seat, the method includes monitoring an occupant in the passenger seat for one or more biometrics indicative of a health condition. This can include using the sensors 104, 106 described above, as indicated in FIG. 4 with the boxes 220 and 230.

Monitoring includes monitoring temperature of the occupant and outputting an alert if the temperature of the occupant is outside of a predetermined range, as indicated in FIG. 4 with boxes 240 and 250. For example, the passenger and/or crew can be alerted if the passenger's body temperature is reading outside the range 35° C. to 37.7° C. Monitoring can also include monitoring images of the occupant and outputting an alert if the images are indicative of a respiratory tract infection, as indicated in boxes 260 and 250 in FIG. 4. If the body temperature and thermal imaging do not indicate a health issue, the system can output a confirmation that the passenger is healthy, as indicated in boxes 270 and 280 in FIG. 4.

Monitoring includes monitoring pulse rate of the occupant, e.g. using the sensor 106 shown in FIG. 2, and outputting an alert if the pulse rate is outside of a predetermined range, e.g. 60 BMP to 100 BPM as indicated in boxes 290 and 300 in FIG. 4. Monitoring can also include monitoring blood oxygen level of the occupant and outputting an alert if the blood oxygen level is outside of a predetermined range, e.g. below 90%, as indicated in boxes 310 and 300 in FIG. 4. If pulse rate and oxygen level are not indicative of a health issue, the system can output a confirmation that the passenger is healthy, as indicated in boxes 320 and 330 in FIG. 4. The monitoring can be repeated during a flight or other transit, as indicated in box 340. The method includes outputting an alert, e.g. to a crewmember using display 132 shown in FIG. 3. The crew member can take this as an indication to take quarantine or other suitable measures for the occupant if the one or more biometrics is/are indicative of a contagious condition.

The methods and systems of the present disclosure, as described above and shown in the drawings, provide for monitoring health of passengers in transit. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A system comprising:
a passenger seat;
a biometric sensor configured to monitor at least one biometric of a passenger seated in the passenger seat; and
a controller operatively connected to control the biometric sensor and to provide output indicative of a health condition of the passenger based on the biometric.

2. The system as recited in claim 1, wherein the passenger seat includes an armrest, and wherein the biometric sensor is mounted in the armrest.

3. The system as recited in claim 1, wherein the passenger seat is a first passenger seat and further comprising a second passenger seat in front of the first passenger seat, wherein the biometric sensor includes an imaging device mounted to the second passenger seat, facing back toward the first passenger seat, configured to monitor the first passenger seat for occupancy and temperature of occupant.

4. The system as recited in claim 3, wherein the first passenger seat includes an armrest, and further comprising a pulse oximeter mounted in the armrest.

5. The system as recited in claim 3, wherein the pulse oximeter is operatively connected to the controller to provide input relating to an occupant's pulse rate, heart rate, blood pressure, oxygen levels and/or vascular state, and wherein the controller is configured to use the input to provide output indicative of an occupant's health state.

6. The system as recited in claim 5, further comprising a display mounted in the headrest of the second passenger seat, facing toward the first passenger seat, and operatively connected to the controller to output information regarding the occupant's health state.

7. The system as recited in claim 5, wherein the armrest includes a display operatively connected to the controller to output information regarding the occupant's health state.

8. The system as recited in claim 5, wherein the first and second passenger seats are in a plurality of passenger seats, each having a respective pulse oximeter and a respective imaging device connected to a controller.

9. The system as recited in claim 8, further comprising a central display operatively connected to the pulse oximeters and imaging devices to alert a crewmember of a health status of any occupant of the passenger seats.

10. A method comprising:
checking a seat for occupancy; and
upon detecting occupancy in the passenger seat, monitoring an occupant in the passenger seat for one or more biometrics indicative of a health condition.

11. The method as recited in claim 10, wherein monitoring includes monitoring temperature of the occupant and outputting an alert if the temperature of the occupant is outside of a predetermined range.

12. The method as recited in claim 10, wherein monitoring includes monitoring images of the occupant and outputting an alert if the images are indicative of a respiratory tract infection.

13. The method as recited in claim 10, wherein monitoring includes monitoring pulse rate of the occupant and outputting an alert if the pulse rate is outside of a predetermined range.

14. The method as recited in claim 10, wherein monitoring includes monitoring blood oxygen level of the occupant and outputting an alert if the blood oxygen level is outside of a predetermined range.

15. The method as recited in claim 10, further comprising outputting an alert to take quarantine measures for the occupant if the one or more biometrics is/are indicative of a contagious condition.
